(19) United States
(12) Patent Application Publication
(10) Pub. No.: US 2009/0240094 A1
(43) Pub. Date: Sep. 24, 2009

(54) Method for the Production of Propene from Propane

(75) Inventors: Sven Crone, Limburgerhof (DE); Otto Machhammer, Mannheim (DE); Götz-Peter Schindler, Mannheim (DE)

Correspondence Address: CONNOLLY BOVE LODGE & HUTZ, LLP, P O BOX 2207, WILMINGTON, DE, 19899, US

(73) Assignee: BASF Aktiengesellschaft Patents, Trademarks and Licenses, Ludwigshafen (DE)

(21) Appl. No.: 11/721,247

(22) PCT Filed: Dec. 8, 2005
(86) PCT No.: PCT/EP2005/013169

§ 371 Date: Jun. 8, 2007

(30) Foreign Application Priority Data
Dec. 9, 2004 (DE) 10 2004 059 355.8

(51) Int. Cl. C07C 5/327

(52) U.S. Cl. 585/655

(57) ABSTRACT

The invention relates to a process for preparing propene from propane, comprising the steps:
A) a feed gas stream a comprising propane is provided;
B) the feed gas stream a comprising propane and an oxygenous gas stream are fed into a dehydrogenation zone and propane is subjected to a nonoxidative catalytic, autothermal dehydrogenation to propene to obtain a product gas stream b comprising propane, propene, methane, ethane, ethene, C₄⁺ hydrocarbons, carbon monoxide, carbon dioxide, steam and hydrogen,
C) the product gas stream b is cooled and steam is removed by condensation to obtain a steam-depleted product gas stream c,
D) carbon dioxide is removed by gas scrubbing to obtain a carbon dioxide-depleted product gas stream d,
E) the product gas stream d is cooled and a liquid hydrocarbon stream e1 comprising propane, propene, methane, ethane, ethene and C₄⁺ hydrocarbons is removed by condensation to leave a residual gas stream e2 comprising methane, hydrogen and carbon monoxide,
F) the liquid hydrocarbon stream e1 is fed into a first distillation zone and separated distillatively into a stream f1 comprising propane, propene and the C₄⁺ hydrocarbons and a stream f2 comprising ethane and ethene,
G) the stream f1 is fed into a second distillation zone and separated distillatively into a product stream g1 comprising propene and a stream g2 comprising propane and the C₄⁺ hydrocarbons.

The invention relates to a process for preparing propene from propane.

Propene is obtained on the industrial scale by dehydrogenating propane.

In the process, known as the UOP-oleflex process, for dehydrogenating propane to propene, a feed gas stream comprising propane is preheated to 600-700° C. and dehydrogenated in a moving bed dehydrogenation reactor over a catalyst which comprises platinum on alumina to obtain a product gas stream comprising predominantly propane, propene and hydrogen, In addition, low-boiling hydrocarbons formed by cracking (methane, ethane, ethene) and small amounts of high boilers (C₄⁺ hydrocarbons) are present in the product gas stream. The product gas mixture is cooled and compressed in a plurality of stages. Subsequently, the C₂ and C₃ hydrocarbons and the high boilers are removed from the hydrogen and methane formed in the dehydrogenation by condensation in a “cold box”. The liquid hydrocarbon condensate is subsequently separated by distillation by removing the C₂ hydrocarbons and remaining methane in a first column and separating the C₃ hydrocarbon stream into a propene fraction having high purity and a propane fraction which also comprises the C₄⁺ hydrocarbons in a second distillation column.

A disadvantage of this process is the loss of C₃ hydrocarbons by the condensation in the cold box. Owing to the large amounts of hydrogen formed in the dehydrogenation and as a consequence of the phase equilibrium, relatively large amounts of C₃ hydrocarbons are also discharged with the hydrogen/methane offgas stream unless condensation is effected at very low temperatures. Thus, it is necessary to work at temperatures of from −20 to −120° C. in order to limit the loss of C₃ hydrocarbons which are discharged with the hydrogen/methane offgas stream.

It is an object of the present invention to provide an improved process for dehydrogenating propane to propene.

The object is achieved by a process for preparing propene from propane, comprising the steps:
A) a feed gas stream a comprising propane is provided;
B) the feed gas stream a comprising propane and an oxygenous gas stream are fed into a dehydrogenation zone and propane is subjected to a nonoxidative catalytic, autothermal dehydrogenation to propene to obtain a product gas stream b comprising propane, propene, methane, ethane, ethene, C₄⁺ hydrocarbons, carbon monoxide, carbon dioxide, steam and hydrogen;
C) the product gas stream b is cooled and steam is removed by condensation to obtain a steam-depleted product gas stream c;
D) carbon dioxide is removed by gas scrubbing to obtain a carbon dioxide-depleted product gas stream d;
E) the product gas stream d is cooled and a liquid hydrocarbon stream e1 comprising propane, propene, methane, ethane, ethene and C₄⁺ hydrocarbons is removed by condensation to leave a residual gas stream e2 comprising methane, hydrogen and carbon monoxide;
F) the liquid hydrocarbon stream e1 is fed into a first distillation zone and separated distillatively into a stream f1 comprising propane, propene and the C₄⁺ hydrocarbons and a stream f2 comprising ethane and ethene;
(G) the stream f1 is fed into a second distillation zone and separated distillatively into a product stream g1 comprising propene and a stream g2 comprising propane and the C₄⁺ hydrocarbons.

In a first process part, A, a feed gas stream a comprising propane is provided. This generally comprises at least 80% by volume of propane, preferably 90% by volume of propane. In addition, the propane-containing feed gas stream A generally also comprises butanes (n-butane, isobutane). Typical compositions of the propane-containing feed gas stream are disclosed in DE-A 102 46 119 and DE-A 102 45 585. Typically, the propane-containing feed gas stream a is obtained from liquid petroleum gas (LPG).

In one process part, B, the feed gas stream comprising propane is fed into a dehydrogenation zone and subjected to a nonoxidative catalytic dehydrogenation. In this process part, propane is dehydrogenated partially in a dehydrogenation reactor over a dehydrogenation-active catalyst to give propene. In addition, hydrogen and small amounts of methane, ethane, ethene and C₄⁺ hydrocarbons (n-butane, isobutane, butenes, butadiene) are obtained. Also obtained in the product gas mixture of the nonoxidative catalytic, autothermal propane dehydrogenation are carbon oxides (CO, CO₂), in particular CO₂, water and inert gases to a small degree. Inert gases (nitrogen) are introduced with the oxygen stream used in the autothermal dehydrogenation when pure oxygen is not fed in. In addition, unconverted propane is present in the product gas mixture.

The nonoxidative catalytic propane dehydrogenation is carried out autothermally. To this end, oxygen is additionally admixed with the reaction gas mixture of the propane dehydrogenation in at least one reaction zone and the hydrogen and/or hydrocarbon present in the reaction gas mixture is at least partly combusted, which directly generates in the reaction gas mixture at least some of the heat required for dehydrogenation in the at least one reaction zone.

One feature of the nonoxidative method compared to an oxidative method is the at least intermediate formation of hydrogen. In the oxidative dehydrogenation, free hydrogen is not formed in substantial amounts.

The nonoxidative catalytic propane dehydrogenation may in principle be carried out in any reactor types known from the prior art. A comparatively comprehensive description of reactor types suitable in accordance with the invention is also contained in “Catalytica® Studies Division, Oxidative Dehydrogenation and Alternative Dehydrogenation Processes” (Study Number 4192 OD, 1993, 430 Ferguson Drive, Mountain View, Calif., 94043-5272, USA).

A suitable reactor form is the fixed bed tubular or tube bundle reactor. In these reactors, the catalyst (dehydrogenation catalyst and if appropriate a specialized oxidation catalyst) is disposed as a fixed bed in a reaction tube or in a bundle of reaction tubes. Customary reaction tube internal diameters are from about 10 to 15 cm. A typical dehydrogenation tube bundle reactor comprises from about 300 to 1000 reaction tubes. The internal temperature in the reaction tubes typically varies in the range from 300 to 1200° C., preferably in the range from 500 to 1000° C. The working pressure is customarily from 0.5 to 8 bar, frequently from 1 to 2 bar, when a low steam dilution is used, or else from 3 to 8 bar when a high steam dilution is used (corresponding to the steam active reforming process (STAR process) or the Linde process) for the dehydrogenation of propane or butane of Phillips Petroleum Co. Typical gas hourly space velocities (GHSV) are from 500 to 2000 h⁻¹, based on hydrocarbon used. The catalyst geometry may, for example, be spherical or cylindrical (hollow or solid).

The nonoxidative catalytic, autothermal propane dehydrogenation may also be carried out under heterogeneous catalysis in a fluidized bed, according to the Snamprogetti/Yarsintez-FBD process. Appropriately, two fluidized beds are operated in parallel, of which one is generally in the state of regeneration. The working pressure is typically from 1 to 2 bar, the dehydrogenation temperature generally from 550 to 600° C. The heat required for the dehydrogenation can be introduced into the reaction system by preheating the dehydrogenation catalyst to the reaction temperature. The admixing of a cofeed comprising oxygen allows the preheater to be dispensed with and the required heat to be generated directly in the reactor system by combustion of hydrogen and/or hydrocarbons in the presence of oxygen. If appropriate, a cofeed comprising hydrogen may additionally be admixed.

The nonoxidative catalytic, autothermal propane dehydrogenation is preferably carried out in a tray reactor. This reactor comprises one or more successive catalyst beds. The number of catalyst beds may be from 1 to 20, advantageously from 1 to 6, preferably from 1 to 4 and in particular from 1 to 3. The catalyst beds are preferably flowed through radially or axially by the reaction gas. In general, such a tray reactor is operated using a fixed catalyst bed. In the simplest case, the fixed catalyst beds are disposed axially in a shaft furnace reactor or in the annular gaps of concentric cylindrical grids. A shaft furnace reactor corresponds to one tray. The performance of the dehydrogenation in a single shaft furnace reactor corresponds to one embodiment. In a further, preferred embodiment, the dehydrogenation is carried out in a tray reactor having 3 catalyst beds.

In general, the amount of the oxygenous gas added to the reaction gas mixture is selected in such a way that the amount of heat required for the dehydrogenation of the propane is generated by the combustion of the hydrogen present in the reaction gas mixture and of any hydrocarbons present in the reaction gas mixture and/or of carbon present in the form of coke. In general, the total amount of oxygen supplied, based on the total amount of propane, is from 0.001 to 0.5 mol/mol, preferably from 0.005 to 0.25 mol/mol, more preferably from 0.05 to 0.25 mol/mol. Oxygen may be used either in the form of pure oxygen or in the form of oxygenous gas which comprises inert gases. In order to prevent high propane and propene losses in the workup (see below), it is essential, however, that the oxygen content of the oxygenous gas used is high and is at least 50% by volume, preferably at least 80% by volume, more preferably at least 90% by volume. A particularly preferred oxygenous gas is oxygen of technical-grade purity with an O₂ content of approx. 99% by volume.

The hydrogen combusted to generate heat is the hydrogen formed in the catalytic propane dehydrogenation and also any hydrogen additionally added to the reaction gas mixture as hydrogenous gas. The amount of hydrogen present should preferably be such that the molar H₂/O₂ ratio in the reaction gas mixture immediately after the oxygen is fed in is from 1 to 10 mol/mol, preferably from 2 to 5 mol/mol. In multistage reactors, this applies to every intermediate feed of oxygenous and any hydrogenous gas.

The hydrogen is combusted catalytically. The dehydrogenation catalyst used generally also catalyzes the combustion of the hydrocarbons and of hydrogen with oxygen, so that in principle no specialized oxidation catalyst is required apart from it. In one embodiment, operation is effected in the presence of one or more oxidation catalysts which selectively catalyze the combustion of hydrogen with oxygen in the presence of hydrocarbons. The combustion of these hydrocarbons with oxygen to give CO, CO₂ and water therefore proceeds only to a minor extent. The dehydrogenation catalyst and the oxidation catalyst are preferably present in different reaction zones.

When the reaction is carried out in more than one stage, the oxidation catalyst may be present only in one, in more than one or in all reaction zones.

Preference is given to disposing the catalyst which selectively catalyzes the oxidation of hydrogen at the points where there are higher partial oxygen pressures than at other points in the reactor, in particular near the feed point for the oxygenous gas. The oxygenous gas and/or hydrogenous gas may be fed in at one or more points in the reactor.

In one embodiment of the process according to the invention, there is intermediate feeding of oxygenous gas and of hydrogenous gas upstream of each tray of a tray reactor. In a further embodiment of the process according to the invention, oxygenous gas and hydrogenous gas are fed in upstream of each tray except the first tray. In one embodiment, a layer of a specialized oxidation catalyst is present downstream of every feed point, followed by a layer of the dehydrogenation catalyst. In a further embodiment, no specialized oxidation catalyst is present. The dehydrogenation temperature is generally from 400 to 1100° C.; the pressure in the last catalyst bed of the tray reactor is generally from 0.2 to 5 bar, preferably from 1 to 3 bar. The GHSV is generally from 500 to 2000 h⁻¹, and, in high-load operation, even up to 100 000 h⁻¹, preferably from 4000 to 16 000 h⁻¹.

A preferred catalyst which selectively catalyzes the combustion of hydrogen comprises oxides and/or phosphates selected from the group consisting of the oxides and/or phosphates of germanium, tin, lead, arsenic, antimony and bismuth. A further preferred catalyst which catalyzes the combustion of hydrogen comprises a noble metal of transition group VIII and/or I of the periodic table.

The dehydrogenation catalysts used generally comprise a support and an active composition. The support generally consists of a heat-resistant oxide or mixed oxide. The dehydrogenation catalysts preferably comprise a metal oxide which is selected from the group consisting of zirconium dioxide, zinc oxide, aluminum oxide, silicon dioxide, titanium dioxide, magnesium oxide, lanthanum oxide, cerium oxide and mixtures thereof, as a support. The mixtures may be physical mixtures or else chemical mixed phases such as magnesium aluminum oxide or zinc aluminum oxide mixed oxides. Preferred supports are zirconium dioxide and/or silicon dioxide, and particular preference is given to mixtures of zirconium dioxide and silicon dioxide.

The active composition of the dehydrogenation catalysts generally comprises one or more elements of transition group VIII of the periodic table, preferably platinum and/or palladium, more preferably platinum. Furthermore, the dehydrogenation catalysts may comprise one or more elements of main group I and/or II of the periodic table, preferably potassium and/or cesium. The dehydrogenation catalysts may further comprise one or more elements of transition group III of the periodic table including the lanthanides and actinides, preferably lanthanum and/or cerium. Finally, the dehydrogenation catalysts may comprise one or more elements of main group III and/or IV of the periodic table, preferably one or more elements from the group consisting of boron, gallium, silicon, germanium, tin and lead, more preferably tin.

In a preferred embodiment, the dehydrogenation catalyst comprises at least one element of transition group VIII, at least one element of main group I and/or II, at least one element of main group III and/or IV and at least one element of transition group III including the lanthanides and actinides.

For example, all dehydrogenation catalysts which are disclosed by WO 99/46039, U.S. Pat. No. 4,788,371, EP-A 705 136, WO 99/29420, U.S. Pat. No. 5,220,091, U.S. Pat. No. 5,430,220, U.S. Pat. No. 5,877,369, EP0 117 146, DE-A 199 37 106, DE-A 199 37 105 and DE-A 199 37 107 may be used in accordance with the invention. Particularly preferred catalysts for the above-described variants of autothermal propane dehydrogenation are the catalysts according to examples 1, 2, 3 and 4 of DE-A 199 37 107.

Preference is given to carrying out the autothermal propane dehydrogenation in the presence of steam. The added steam serves as a heat carrier and supports the gasification of organic deposits on the catalysts, which counteracts carbonization of the catalysts and increases the onstream time of the catalysts. This converts the organic deposits to carbon monoxide, carbon dioxide and in some cases water.

The dehydrogenation catalyst may be regenerated in a manner known per se. For instance, steam may be added to the reaction gas mixture or a gas comprising oxygen may be passed from time to time over the catalyst bed at elevated temperature and the deposited carbon burnt off. The dilution with steam shifts the equilibrium toward the products of dehydrogenation. After the regeneration, the catalyst is reduced with a hydrogenous gas if appropriate.

In the autothermal propane dehydrogenation, a gas mixture is obtained which generally has the following composition: from 10 to 45% by volume of propane, from 5 to 40% by volume of propene, from 0 to 5% by volume of methane, ethane, ethene and C₄⁺ hydrocarbons, from 0 to 5% by volume of carbon dioxide, from 0 to 20% by volume of steam and from 0 to 25% by volume of hydrogen, and also from 0 to 5% by volume of inert gases.

When it leaves the dehydrogenation zone, the product gas stream b is generally under a pressure of from 1 to 5 bar, preferably from 1.5 to 3 bar, and has a temperature in the range from 400 to 700° C.

The product gas stream b may be separated into two substreams, in which case one substream is recycled into the autothermal dehydrogenation, corresponding to the cycle gas method described in DE-A 102 11 275 and DE-A 100 28 582.

In process part C, water is initially removed from the product gas stream b. The removal of water is carried out by condensation, by cooling and if appropriate compressing the product gas stream b, and may be carried out in one or more cooling and if appropriate compression stages. In general, the product gas stream b is cooled for this purpose to a temperature in the range from 30 to 80° C., preferably from 40 to 65° C. In addition, the product gas stream may be compressed, for example to a pressure in the range from 5 to 25 bar.

In one embodiment of the process according to the invention, the product gas stream b is passed through a battery of heat exchangers and initially thus initially cooled to a temperature in the range from 50 to 200° C. and subsequently cooled further in a quenching tower with water to a temperature of from 40 to 80° C., for example 55° C. This condenses out the majority of the steam, but also some of the C₄⁺ hydrocarbons present in the product gas stream b, in particular the C₅⁺ hydrocarbons.

A steam-depleted product gas stream c is obtained. This generally still comprises from 0 to 5% by volume of steam. For the virtually full removal of water from the product gas stream c, a drying by means of molecular sieve may be provided.

In one process step, D), carbon dioxide is removed from the product gas stream c by gas scrubbing to obtain a carbon dioxide-depleted product gas stream d. The carbon dioxide gas scrubbing may be preceded by a separate combustion stage in which carbon monoxide is oxidized selectively to carbon dioxide.

For the CO₂ removal, the scrubbing liquid used is generally sodium hydroxide solution, potassium hydroxide solution or an alkanolamine solution; preference is given to using an activated N-methyldiethanolamine solution. In general, before the gas scrubbing is carried out, the product gas stream c is compressed to a pressure in the range from 5 to 25 bar by compression in one or more stages.

A carbon dioxide-depleted product gas stream d having a CO₂ content of generally <100 ppm, preferably <10 ppm, is obtained.

In one process step, E), the product gas stream d is cooled and a liquid hydrocarbon stream e1 comprising propane, propene, methane, ethane, ethene and C₄⁺ hydrocarbons is removed by condensation to leave a residual gas stream e2 comprising methane, hydrogen and carbon monoxide. To this end, the product gas stream d is generally compressed to a pressure in the range from 5 to 25 bar and cooled to a temperature in the range from −10 to −120° C. The compression may be effected in a plurality of stages, for example in two or three stages; it is preferably effected in a plurality of stages, for example three stages. In one embodiment of the process according to the invention, before the scrubbing step C) is carried out, a one- or two-stage compression of the product gas stream c is effected to a pressure in the range from 5 to 12 bar and subsequently a one- or two-stage compression of the product gas stream d to a pressure in the range from 10 to 25 bar. The cooling too may be effected in a plurality of stages; it is preferably effected in a plurality of stages. Suitable coolants are ethene, propene and propane which are cooled to temperatures in the range from −40° C. to −100° C. by compression to pressures up to 20 bar and subsequent decompression.

In general, the product gas stream is cooled by heat exchange with a coolant in a “cold box”. The cooling may be effected in a plurality of stages using a plurality of cooling circuits. The cooling may be effected in a plurality of stages in one column, in which case the gas rising in the column is withdrawn, cooled, (partly) condensed and recycled into the column. At the bottom of the column, the condensate is withdrawn, at the top of the column the uncondensed gas which has also not condensed in the uppermost cooling circuit.

The low hydrogen content of the product gas stream d as a consequence of the performance of the dehydrogenation B) as an autothermal dehydrogenation with simultaneous combustion of the hydrogen formed results in the C₃ hydrocarbons being very predominantly condensed out in the removal step E) and only a very small portion of the C₃ hydrocarbons being discharged with the hydrogen/methane offgas stream.

A liquid hydrocarbon condensate stream e1 is obtained which comprises generally from 20 to 60 mol % of propane, from 20 to 60 mol % of propene, from 0 to 5 mol % of methane, from 0 to 5 mol % of ethane and ethene, and from 0 to 5 mol % of C₄⁺ hydrocarbons.

Before step E) is carried out, a drying stage is carried out in which the product gas stream d is dried by passing it over a molecular sieve. Suitable molecular sieves are known to those skilled in the art. In general, the drying stage is carried out after the last compression stage, immediately before the condensation step E). The temperature of the product gas stream d to be dried is generally from 0 to 50° C., preferably from 10 to 30° C.

After the drying step has been carried out, the water content of the gas stream d is <500 ppm, preferably <100 ppm.

In one process step, F, the liquid hydrocarbon stream e1 is fed into a first distillation zone and separated distillatively into a stream f1 comprising the C₃ hydrocarbons propane, propene and the C₄⁺ hydrocarbons and a stream f2 comprising the C₂ hydrocarbons ethane and ethene. To this end, the hydrocarbon stream e1 is generally fed into a C2/C3 separating column having typically from 20 to 60 theoretical plates, for example approx. 30 theoretical plates. This column is generally operated at a pressure in the range from 12 to 30 bar, for example at approx. 25 bar. The bottom temperature is generally from 40 to 100° C., for example approx. 60° C., the top temperature from −10 to 10° C., for example approx. 10° C.

A stream f1 composed of propane, propene and the C₄⁺ hydrocarbons is obtained as the bottom draw stream having a total ethane/ethene content of generally <5000 ppm, preferably <1000 ppm, more preferably <500 ppm.

In one process step, G, the liquid hydrocarbon stream f1 is fed into a first distillation zone and separated distillatively into a stream g1 comprising propene and a stream g2 comprising propane and the C₄⁺ hydrocarbons. To this end, the hydrocarbon stream f1 is generally fed into a C3 separating column (“C3 splitter”) having typically from 80 to 200 theoretical plates, for example approx. 100 theoretical plates. This column is generally operated at a pressure in the range from 15 to 30 bar, for example at approx. 25 bar. The bottom temperature is generally from 40 to 100° C., for example approx. 68° C., the top temperature from 30 to 60° C., for example approx. 60° C.

The invention is illustrated in detail below with reference to the drawing.

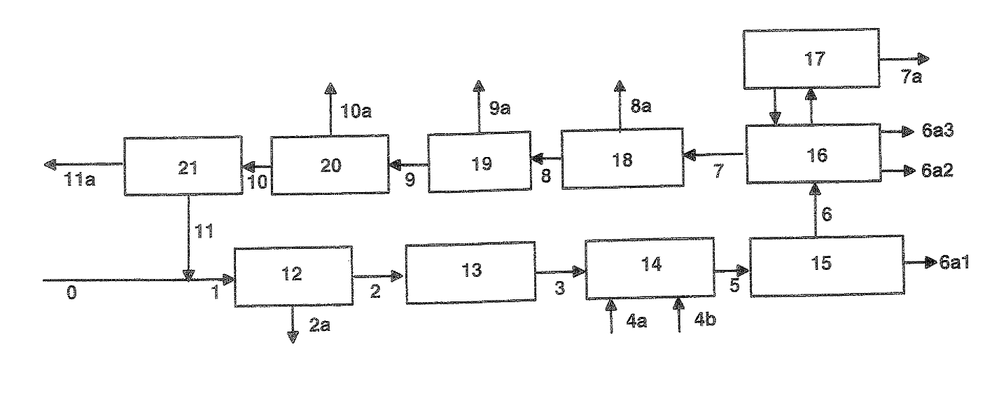

FIG. 1 shows a schematic of one embodiment of the process according to the invention.

Fresh propane 0 which consists, for example, to an extent of 98% by volume of propane and additionally 2% by volume of C₄⁺ hydrocarbons (mainly butane) is fed together with the recycle stream from the C3 separation column 11 composed of propane and propene into a C3/C4 separation column 12, and separated distillatively into a stream 2a composed of C₄⁺ hydrocarbons 2a which is obtained as the bottom draw stream and a stream 2 having a propane content of >99% by volume which is obtained as the top draw stream.

The propane stream 2 is preheated to a temperature of approx. 450° C. in a preheater 13, for example by heat exchange with the product gas stream 5, and from there enters the dehydrogenation reactor 14 at this temperature. The dehydrogenation reactor 3 is preferably configured as a radial flow reactor. A steam stream 4a and an oxygen stream 4b composed of oxygen of technical-grade purity having an oxygen content of approx. 99% by volume are additionally fed into this reactor. The product gas mixture 5 leaves the reactor at a temperature of approx. 600° C. and consists of propane, propene, methane, ethane, ethene, C₄ hydrocarbons, carbon dioxide, steam and hydrogen, and also small amounts of nitrogen. The product gas stream 3 is cooled to a temperature of approx. 55° C. in a condenser battery. In the condenser battery 6, the majority of the steam condenses out and is drawn off as liquid condensate 6a1.

The stream 6 is compressed from approx. 2 bar to approx. 15 bar in a three-stage compressor with intermediate cooling. This condenses out further amounts of steam which are drawn off as liquid condensate 6a2 and 6a3. Between the second and third compressor stage, carbon dioxide is removed by means of gas scrubbing. To this end, the compressed gas stream is passed into a scrubbing column 17 and contacted with an activated N-methyldiethanolamine solution as the scrubbing liquid From the laden scrubbing liquid, carbon dioxide is released again by heating in a desorption column and the scrubbing liquid is regenerated.

The gas stream 7 which has been compressed to approx. 15 bar and freed of carbon dioxide is precooled to approx. 10° C., fed to a drying stage 18 and freed there of water traces by means of a molecular sieve. To this end, 2 parallel apparatuses filled with molecular sieves are used, of which one is always operated in absorption mode and the other in regeneration mode. The dried gas stream 8 enters the “cold box” at a temperature of approx. 10° C., where ethane, ethene, propane, propene, the C₄⁺ hydrocarbons and some of the methane condense out at −40° C. as a liquid hydrocarbon mixture 9. This leaves an offgas stream 9a which comprises a small portion of the C₂ hydrocarbons present in the gas stream 8, the majority of the methane present in the gas stream 8 and virtually all of the hydrogen present in the gas stream 8.

The liquid hydrocarbon mixture 9 is separated in a C2/C3 separation column 20 distillatively into a stream 10 composed of propane, propene and C₄⁺ hydrocarbons and a stream 10a composed of methane, ethane and ethene. The stream 10 is fed to a C3 separation column 21 and separated into a product stream 11a composed of propene having a purity of 99.5% and the recycle stream 11 which consists predominantly of propane and additionally C₄⁺ hydrocarbons and also small amounts of propene (approx. 1% by weight).

The invention is illustrated in detail by the example which follows.

### EXAMPLES

The variant, shown in the FIGURE, of the process according to the invention was simulated by calculation. The process parameters below were assumed.

### Example 1

A capacity of the plant of 350 kt/a at running time 8000 h, corresponding to 43 750 kg/h of propene, is assumed.

In addition to 98% by weight of propane, the fresh propane comprises 2% by weight of butane. The butane content is depleted to 0.01% by weight in a C3/C4 separation column having 40 theoretical plates at operating pressure 10 bar and a reflux ratio of 0.41.

The propane stream is preheated to 450° C., enters the dehydrogenation zone and is subjected to an autothermal dehydrogenation. The conversion of the dehydrogenation, based on propane, is 50%, the selectivity of propene formation is 90%. In addition, 5% cracking products and 5% CO₂ are formed by total combustion. The water concentration in the exit gas of the dehydrogenation zone is 9% by weight; the residual oxygen content in the exit gas is 0% by weight; the exit temperature of the product gas mixture is 600° C.

The exit gas is cooled to 55° C. at 1.8 bar and water is condensed out up to the saturation vapor pressure.

Subsequently, the product gas mixture is compressed in 3 stages. In the first compressor stage, the mixture is compressed from 1.8 bar to 4.5 bar, in the second compressor stage from 4.5 to 11 bar and in the third compressor stage from 11 bar to 18 bar. Downstream of each compressor stage, the gas mixture is cooled to 55° C. After the second compressor stage, CO₂ is removed fully from the gas stream by gas scrubbing. After the third compressor stage, the residual water is removed fully from the gas stream.

Subsequently, the gas stream is cooled to −40° C. at 18 bar. This condenses out the C₂⁻, C₃⁻ and C₄⁺ hydrocarbons.

The liquid hydrocarbon condensate is separated in a C2/C3 separation column having 30 theoretical plates at 25 bar and a reflux ratio of 0.74. The bottom temperature is 62° C., the top temperature 10° C. The residual ethane content of the bottom product is 0.01% by weight.

The bottom effluent is fed to a propane/propene separation column having 100 theoretical plates which is operated at 25 bar with a reflux ratio of 30. The bottom temperature is 60° C., the top temperature 68° C. At the top, propene is obtained with a purity of 99.5% by weight.

### Comparative Example 1

Instead of the autothermal propane dehydrogenation, an isothermal propane dehydrogenation is carried out.

To this end, the propane stream is preheated to 450° C., enters the dehydrogenation zone and is subjected to an isothermal dehydrogenation. The water content of the entry gas is 50% by weight. The conversion of the dehydrogenation is, based on propane, 50%; the selectivity of propene formation is 90%. In addition, cracking products are formed to an extent of 8% and CO₂ to an extent of 2%. The temperature of the exit gas is 600° C. Tables 1 (example) and 2 (comparative example) reproduce the amounts (in kg/h) and composition (in parts by mass, Σ=1.0) of the gas streams according to the FIGURE. As can be taken from the tables, the amount of C₃ hydrocarbons (propane and propene) which is discharged with the hydrogen stream 9a is very much higher for the comparative example than for the example.

EXAMPLE 1

| Stream No. | 0 | 1 | 2a | 2 | 3 | 4a | 4b | 5 |
|---|---|---|---|---|---|---|---|---|
| Amount [kg/h] | 55079.1587 | 106604.5642 | 1111.5339 | 105493.0307 | 105493.0307 | 104.9672 | 14686.0095 | 120283.3255 |
| BUTANE | 0.02 | 0.0104 | 0.99 | 0.0001 | 0.0001 | 0 | 0 | 0.0001 |
| PROPANE | 0.98 | 0.9847 | 0.01 | 0.9950 | 0.9950 | 0 | 0 | 0.4363 |
| PROPENE | 0 | 0.0048 | 0 | 0.0049 | 0.0049 | 0 | 0 | 0.3790 |
| WATER | 0 | 0 | 0 | 0 | 0 | 1 | 0 | 0.0924 |
| ETHENE | 0 | 0 | 0 | 0 | 0 | 0 | 0 | 0.0069 |
| ETHANE | 0 | 0 | 0 | 0 | 0 | 0 | 0 | 0.0149 |
| CO2 | 0 | 0 | 0 | 0 | 0 | 0 | 0 | 0.0435 |
| H2 | 0 | 0 | 0 | 0 | 0 | 0 | 0 | 0.0117 |
| O2 | 0 | 0 | 0 | 0 | 0 | 0 | 0.99 | 0 |
| N2 | 0 | 0 | 0 | 0 | 0 | 0 | 0.01 | 0.0012 |
| CO | 0 | 0 | 0 | 0 | 0 | 0 | 0 | 0.0139 |
| Temperature [° C.] | 20 | 50 | 78.14 | 26.93 | 450 | 350 | 600 | 600 |
| Pressure [bar] | 10 | 10 | 10 | 10 | 10 | 1 | 1.8 | 1.8 |

| Stream No. | 6 | 6a1 | 6a2 | 6a3 | 7a | 7 | 8a | 8 |
|---|---|---|---|---|---|---|---|---|
| Amount [kg/h] | 114760.3846 | 5522.9402 | 3490.4776 | 1277.7084 | 5238.1258 | 104754.0750 | 845.4069 | 103908.67 |
| BUTANE | 0.0001 | 0.0001 | 0.0001 | 0.0003 | 0 | 0.0001 | 0 | 0.0001 |
| PROPANE | 0.4573 | 0.0001 | 0.0002 | 0.0005 | 0 | 0.5010 | 0 | 0.5051 |
| PROPENE | 0.3973 | 0.0001 | 0.0004 | 0.0010 | 0 | 0.4352 | 0 | 0.4387 |
| WATER | 0.0488 | 0.9991 | 0.9975 | 0.9938 | 0 | 0.0081 | 1 | 0 |
| ETHENE | 0.0073 | 0.0000 | 0.0000 | 0.0000 | 0 | 0.0080 | 0 | 0.0080 |
| ETHANE | 0.0156 | 0.0007 | 0.0018 | 0.0044 | 0 | 0.0169 | 0 | 0.0171 |
| CO2 | 0.0456 | 0 | 0 | 0 | 1 | 0 | 0 | 0 |
| H2 | 0.0123 | 0 | 0 | 0 | 0 | 0.0134 | 0 | 0.0135 |
| O2 | 0 | 0 | 0 | 0 | 0 | 0 | 0 | 0 |
| N2 | 0.0013 | 0 | 0 | 0 | 0 | 0.0014 | 0 | 0.0014 |
| CO | 0.0145 | 0 | 0 | 0 | 0 | 0.0159 | 0 | 0.0160 |
| Temperature [° C.] | 55 | 55 | 55 | 55 | 55 | 30 | 40.69 | 40.69 |
| Pressure [bar] | 1.8 | 1.8 | 4.5 | 11 | 11 | 18.5 | 18.5 | 18.5 |

| Stream No. | 9 | 9a | 10 | 10a | 11a | 11 |
|---|---|---|---|---|---|---|
| Amount [kg/h] | 97841.54 | 6067.1312 | 95275.41 | 2566.1305 | 43750.0001 | 51525.4055 |
| BUTANE | 0.0001 | 0.0000 | 0.0001 | 0 | 0 | 0.0002 |
| PROPANE | 0.5246 | 0.1908 | 0.5375 | 0.0446 | 0.0048 | 0.9898 |
| PROPENE | 0.4528 | 0.2117 | 0.4623 | 0.1000 | 0.9950 | 0.01 |
| WATER | 0 | 0 | 0 | 0 | 0 | 0 |
| ETHENE | 0.0067 | 0.0297 | 00 | 0.2550 | 0 | 0 |
| ETHANE | 0.0158 | 0.0369 | 0.0001 | 0.6005 | 0.0002 | 0 |
| CO2 | 0 | 00 | 0 | 0 | 0 | 0 |
| H2 | 0 | 0.2319 | 0 | 0 | 0 | 0 |
| O2 | 0 | 0 | 00 | 0 | 0 | 0 |
| N2 | 0 | 0.0242 | 00 | 0 | 0 | 0 |
| CO | 0 | 0.2747 | 00 | 0 | 0 | 0 |
| Temperature [° C.] | −40 | −40 | 62.47 | 10.09 | 59.25 | 68.07 |
| Pressure [bar] | 18.5 | 18.5 | 25 | 25 | 25 | 25 |

Comparative example

| Stream No. | 0 | 1 | 2a | 2 | 3 | 4 | 5 | 6 |
|---|---|---|---|---|---|---|---|---|
| Amount [kg/h] | 56645.2497 | 108336.021 | 1140.3146 | 107195.707 | 107195.707 | 106668.079 | 217346.859 | 115141.928 |
| BUTANE | 0.02 | 0.0105 | 0.99 | 0.0001 | 0.0001 | 0 | 0.0000 | 0.0001 |
| PROPANE | 0.98 | 0.9847 | 0.01 | 0.9951 | 0.9951 | 0 | 0.2454 | 0.4631 |
| PROPENE | 0 | 0.0048 | 0 | 0.0048 | 0.0048 | 0 | 0.2131 | 0.4022 |
| WATER | 0 | 0 | 0 | 0 | 0 | 1 | 0.4988 | 0.0545 |
| ETHENE | 0 | 0 | 0 | 0 | 0 | 0 | 0.0094 | 0.0177 |
| ETHANE | 0 | 0 | 0 | 0 | 0 | 0 | 0.0067 | 0.0122 |
| CO2 | 0 | 0 | 0 | 0 | 0 | 0 | 0.0098 | 0.0185 |
| METHANE | 0 | 0 | 0 | 0 | 0 | 0 | 0.0036 | 0.0067 |
| H2 | 0 | 0 | 0 | 0 | 0 | 0 | 0.0101 | 0.0191 |
| O2 | 0 | 0 | 0 | 0 | 0 | 0 | 0 | 0 |
| N2 | 0 | 0 | 0 | 0 | 0 | 0 | 0 | 0 |
| CO | 0 | 0 | 0 | 0 | 0 | 0 | 0.0031 | 0.0059 |
| Temperature [° C.] | 20 | 50 | 78.14 | 26.93 | 450 | 350 | 600 | 55 |
| Pressure [bar] | 10 | 10 | 10 | 10 | 10 | 10 | 1.8 | 1.8 |

| Stream No. | 6a1 | 6a2 | 6a3 | 7a | 7 | 8a | 8 |
|---|---|---|---|---|---|---|---|
| Amount [kg/h] | 102204.929 | 3911.9338 | 1430.6569 | 2129.2010 | 107670.137 | 948.9521 | 106721.18 |
| BUTANE | 0.0000 | 0.0001 | 0.0002 | 0 | 0.0001 | 0 | 0.0001 |
| PROPANE | 0.0001 | 0.0002 | 0.0005 | 0 | 0.4953 | 0 | 0.4997 |
| PROPENE | 0.0001 | 0.0003 | 0.0009 | 0 | 0.4301 | 0 | 0.4339 |
| WATER | 0.9993 | 0.9982 | 0.9954 | 0 | 0.0088 | 1 | 0 |
| ETHENE | 0.0000 | 0.0000 | 0.0000 | 0 | 0.0189 | 0 | 0.0191 |
| ETHANE | 0.0005 | 0.0012 | 0.0031 | 0 | 0.0130 | 0 | 0.0131 |
| CO2 | 0 | 0 | 0 | 1 | 0 | 0 | 0 |
| METHANE | 0 | 0 | 0 | 0 | 0.0072 | 0 | 0.0073 |
| H2 | 0 | 0 | 0 | 0 | 0.0204 | 0 | 0.0206 |
| O2 | 0 | 0 | 0 | 0 | 0 | 0 | 0 |
| N2 | 0 | 0 | 0 | 0 | 0 | 0 | 0 |
| CO | 0 | 0 | 0 | 0 | 0.0063 | 0 | 0.0063 |
| Temperature [° C.] | 55 | 55 | 55 | 55 | 30 | 41.07 | 41.07 |
| Pressure [bar] | 1.8 | 4.5 | 11 | 11 | 18.5 | 18.5 | 18.5 |

| Stream No. | 9 | 9a | 10 | 10a | 11a | 11 |
|---|---|---|---|---|---|---|
| Amount [kg/h] | 98501.836 | 8219.3483 | 95440.7715 | 3061.0647 | 43750 | 51690.771 |
| BUTANE | 0.0001 | 0.0000 | 0.0001 | 0 | 0 | 0.0001 |
| PROPANE | 0.5234 | 0.2149 | 0.5383 | 0.0599 | 0.0048 | 0.9899 |
| PROPENE | 0.4503 | 0.2376 | 0.4615 | 0.1 | 0.995 | 0.0100 |
| WATER | 0 | 0 | 0 | 0 | 0 | 0 |
| ETHENE | 0.0146 | 0.0731 | 0 | 0.4687 | 0 | 0 |
| ETHANE | 0.0116 | 0.0306 | 0.0001 | 0.3714 | 0.0002 | 0 |
| CO2 | 0 | 0 | 0 | 0 | 0 | 0 |
| METHANE | 0 | 0.0944 | 0 | 0 | 0 | 0 |
| H2 | 0 | 0.2670 | 0 | 0 | 0 | 0 |
| O2 | 0 | 0 | 0 | 0 | 0 | 0 |
| N2 | 0 | 0 | 0 | 0 | 0 | 0 |
| CO | 0 | 0.0824 | 0 | 0 | 0 | 0 |
| Temperature [° C.] | −40 | −40 | 62.48 | 8.04 | 59.25 | 50 |
| Pressure [bar] | 18.5 | 18.5 | 25 | 25 | 25 | 10 |

1-8. (canceled)

9. A process for preparing propene from propane, comprising the steps:
A) providing a feed gas stream (a) comprising propane;
B) feeding said feed gas stream (a) and an oxygenous gas stream having an oxygen content of at least 50% by volume into a dehydrogenation zone and nonoxidatively, catalytically, autothermally dehydrogenating propane to obtain a product gas stream (b) comprising propane, propene, methane, ethane, ethene, C₄⁺ hydrocarbons, carbon monoxide, carbon dioxide, steam, and hydrogen;
C) cooling and removing the steam from said product gas stream (b) by condensation to obtain a steam-depleted product gas stream (c);
D) scrubbing said product gas stream (c) to remove carbon dioxide and obtain a carbon dioxide-depleted product gas stream (d);
E) cooling said product gas stream (d) and removing a liquid hydrocarbon stream (e1) comprising propane, propene, methane, ethane, ethene, and C₄⁺ hydrocarbons from said product gas stream (d) by condensation, leaving a residual gas stream (e2) comprising methane, hydrogen and carbon monoxide;
F) feeding said liquid hydrocarbon stream (e1) into a first distillation zone and distillatively separating said liquid hydrocarbon stream (e1) into a stream (f1) comprising propane, propene, and C₄⁺ hydrocarbons, and a stream (f2) comprising ethane and ethene; and
G) feeding said stream (f1) into a second distillation zone and distillatively separating said stream (f1) into a product stream (g1) comprising propene, and a stream (g2) comprising propane and the C₄⁺ hydrocarbons.

10. The process according to claim 9, further comprising the additional step H) of feeding said stream (g2) and fresh propane into a third distillation zone and distillatively separating the resulting combination of said stream (g2) and fresh propane into said feed gas stream (a) and a stream comprising C₄⁺ hydrocarbons.

11. The process according to claim 9, wherein said product gas stream (b) is cooled in step C) to a temperature in the range of from 30° C. to 80° C.

12. The process according to claim 9, wherein said product gas stream (c) is compressed to a pressure of from 5 bar to 25 bar prior to step D).

13. The process according to claim 9, wherein said product gas stream (d) is compressed to a pressure of from 5 bar to 25 bar prior to step E).

14. The process according to claim 9, wherein said product gas stream (d) is cooled to a temperature in the range of from −10° C. to −120° C. in step E).

15. The process according to claim 9, wherein said product gas stream (d) is dried by passing said product gas stream (d) over a molecular sieve prior to step E).

16. The process according to claim 9, wherein said oxygenous gas stream has an oxygen content of at least 90% by volume.
